(19) United States
(12) Patent Application Publication
(10) Pub. No.: US 2004/0265197 A1
(43) Pub. Date: Dec. 30, 2004

(54) Garbage bin with ozone-based air cleaner

(76) Inventor: Tsong-Yow Lin, Taichung Hsien (TW)

Correspondence Address: NIKOLAI & MERSEREAU, P.A., 900 SECOND AVENUE SOUTH, SUITE 820, MINNEAPOLIS, MN, 55402, US

(21) Appl. No.: 10/610,051

(22) Filed: Jun. 30, 2003

(51) Int. Cl. A61L 2/00; A61L 9/00

(52) U.S. Cl. 422/305; 422/292; 422/300; 422/306

(57) ABSTRACT

A garbage storage apparatus capable of generating ozone for cleaning air is disclosed. The garbage storage apparatus includes a bin, a cover and an ozone-based air cleaner. The bin defines a space for storing garbage. The cover is used for covering the bin. The ozone-based air cleaner is communicated with the bin for cleaning air in the bin.

### BACKGROUND OF INVENTION

1. Field of Invention

The present invention relates to a garbage bin with an ozone-based air cleaner.

2. Related Prior Art

Taiwanese Patent Publication No. 483430 discloses a conventional garbage storage apparatus for storing two sorts of garbage. This conventional garbage storage apparatus includes a bin 1 and a holder 2 for holding two bags 3 in the bin 1. The bin 1 includes an upper edge 12 defining an opening. The holder 2 includes a lever 21 and two frames 22. The lever 21 is mounted on an upper edge 12 so as to divide the opening defined in the upper edge 12 into two sub-openings. Each of the bags 3 is put in the bin 1 through one of the sub-openings. Each of the frames 22 is put in an upper edge of one of the bags 3 and then mounted on the upper edge of the bin 1 and the lever 21. Thus, each of the bags 3 is retained open for taking a sort of garbage. In use, one of the bags 3 may store kitchen waste and the other one of the bags 3 other garbage, for example. However, the kitchen waste tends to stink soon after it is put in one of the bags 3.

The present invention is therefore intended to obviate or at least alleviate the problems encountered in prior art.

### SUMMARY OF INVENTION

It is the primary objective of the present invention to provide a garbage storage apparatus for eliminating odor.

According to the present invention, a garbage storage apparatus includes a bin, a cover and an ozone-based air cleaner. The bin defines a space for storing garbage. The cover is used for covering the bin. The ozone-based air cleaner is communicated with the bin for cleaning air in the bin.

Other objects, advantages, and novel features of the invention will become more apparent from the following detailed description when taken in conjunction with the attached drawings.

### BRIEF DESCRIPTION OF DRAWINGS

The present invention will be described through detailed illustration of embodiments referring to the attached drawings wherein:

FIG. 1 is a perspective view of a garbage storage apparatus according to a first embodiment of the present invention.

FIG. 2 is an exploded view of an ozone-based air cleaner used in the garbage storage apparatus shown in FIG. 1.

FIG. 3 is a cross-sectional view of the garbage storage apparatus shown in FIG. 1.

FIG. 4 is an exploded view of an ozone-based air cleaner according to a second embodiment of the present invention.

FIG. 5 is an exploded view of a garbage storage apparatus according to a third embodiment of the present invention.

FIG. 6 is a cross-sectional view of a garbage storage apparatus according to a fourth embodiment of the present invention.

FIG. 7 is an exploded view of a garbage storage apparatus according to a fifth embodiment of the present invention.

FIG. 8 is a cross-sectional view of a garbage storage apparatus according to a sixth embodiment of the present invention.

FIG. 9 is an exploded view of a garbage storage apparatus according to a seventh embodiment of the present invention.

FIG. 10 is an exploded view of an ozone-based air cleaner used in a garbage storage apparatus of FIG. 9.

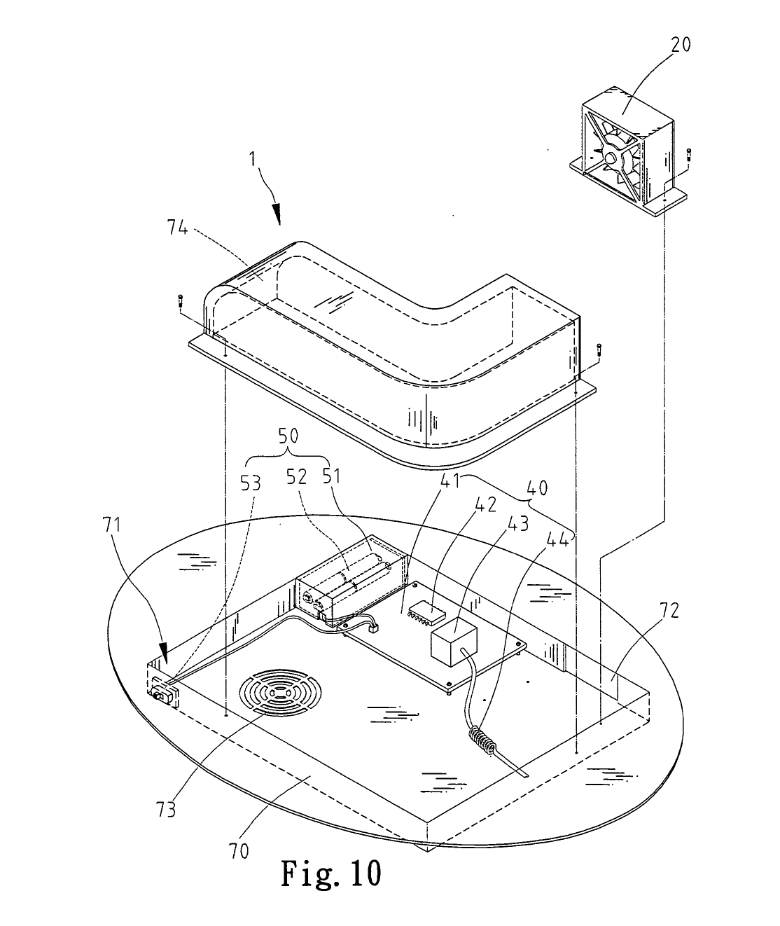

FIG. 11 is an exploded view of an ozone-based air cleaner according to an eighth embodiment of the present invention.

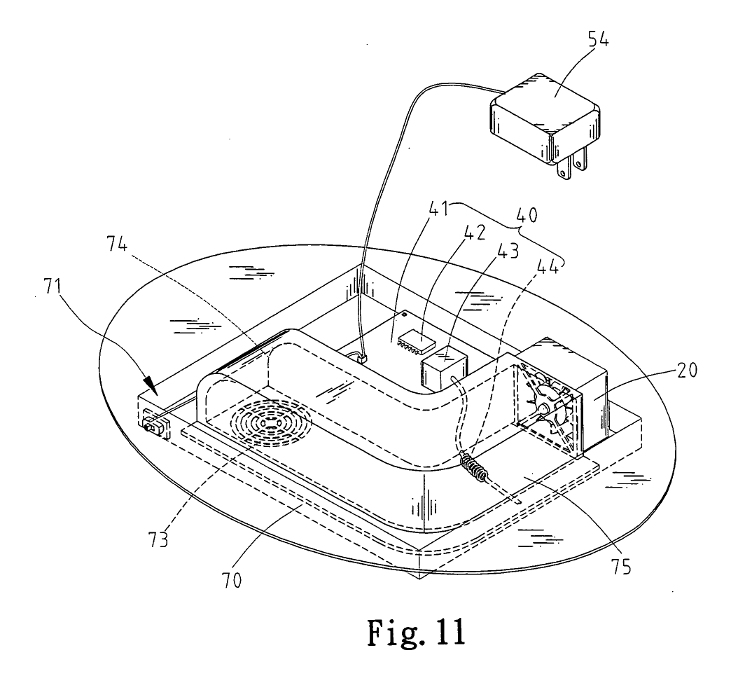

### DETAILED DESCRIPTION OF PREFERRED EMBODIMENT

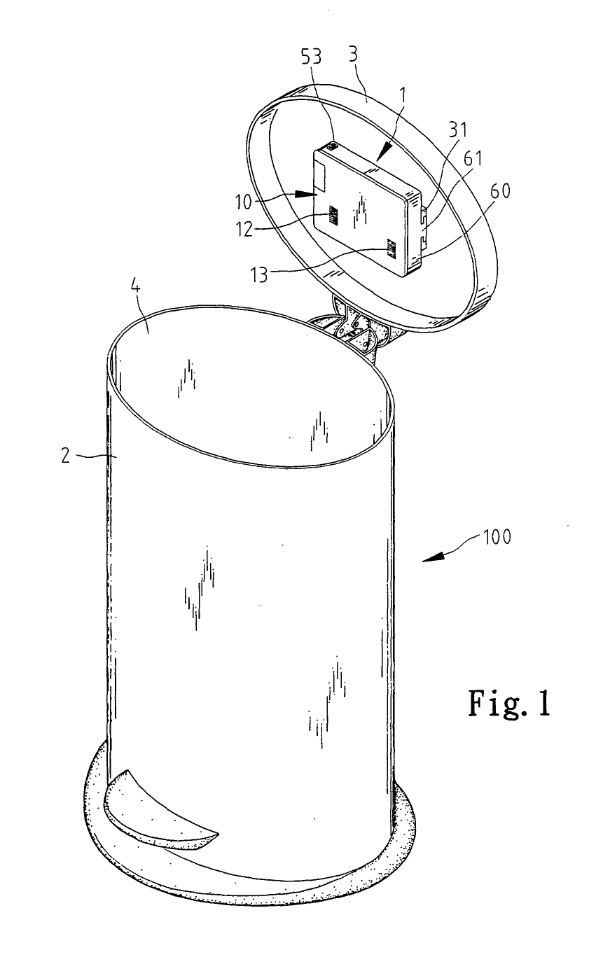

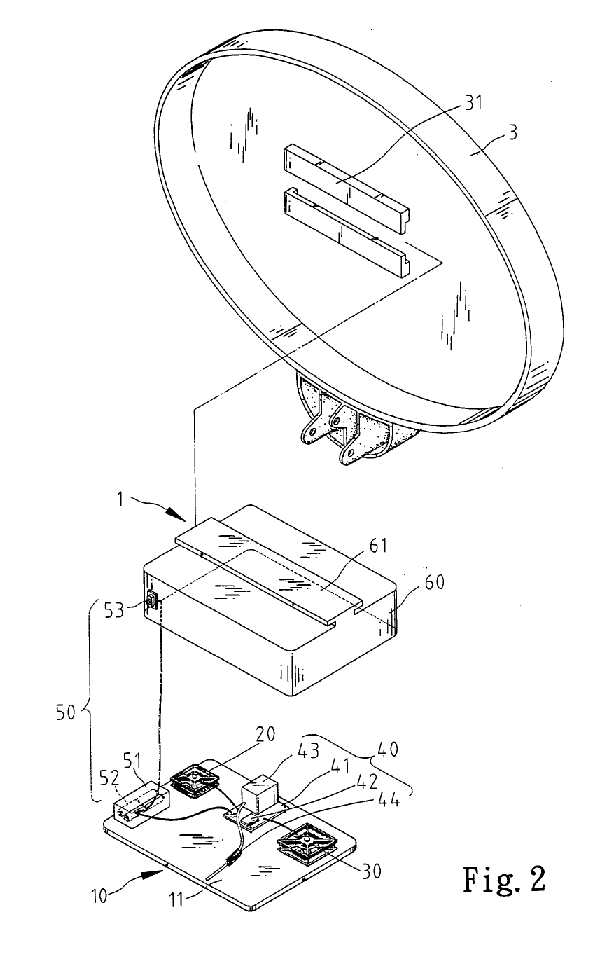

Referring to FIGS. 1-3, according to a first embodiment of the present invention, a garbage storage apparatus 100 includes a bin 2, a cover 3 pivotally mounted on the bin 2 and an ozone-based air cleaner 1 communicated with the bin 2.

The bin 2 defines a space 4 for receiving garbage. In another embodiment, an internal bin (not shown) may be installed in the space 4 in order to receive garbage. However, such an internal bin is optional and can be saved.

A pedal (not numbered) is mounted on the bin 2. The pedal is connected with the cover 3 through a linkage (not shown). Thus, the cover 3 can be lifted via treading the pedal.

The ozone-based air cleaner 1 includes a board 10, a first fan 20, a second fan 30, a ozone generator 40, a power supply 50 and a shell 60.

The board 10 includes an upper face 11 on which the first fan 20, the second fan 30, the light source 40, the power supply 50 and the shell 60 are mounted. The board 10 defines a first passage 12 and a second passage 13. The first passage 12 may consist of one or more holes or slots. The second passage 13 may consist of one or more holes or slots.

The first fan 20 is located over the first passage 12. The first fan 20 can draw air into a space defined by means of the board 10 and the shell 60 through the first passage 12.

The second fan 30 is located over the second passage 13. The second fan 30 can drive air from the space defined by means of the board 10 and the shell 60 through the second passage 13.

The ozone generator 40 includes a circuit board 41 connected with the first fan 20 and the second fan 30, an integrated circuit 42 installed at the circuit board 41, a direct circuit (“DC”) generator 43 installed at the circuit board 41 and a discharger 44 connected with the DC generator 43. The discharger 44 can generate ozone from air flowing past it by means of discharge.

The power supply 50 includes a box 51, two batteries 52 and a switch 53. The box 51 is secured to the board 10. The batteries 52 are put in the box 51 for providing DC to the first fan 20, the second fan 30 and the ozone generator 40. The switch 53 is installed at the shell 60 and connected with the batteries 52.

The cover 60 includes a rail 61 extending on an upper face thereof. The rail 61 is shaped like a rail. Two flanges 31 extend on a lower face of the cover 3. The flanges 31 are shaped in compliance with the rail 61. The rail 61 is engaged with the flanges 31 in a sliding manner. Thus, the ozone-based air cleaner 1 is attached to the cover 3.

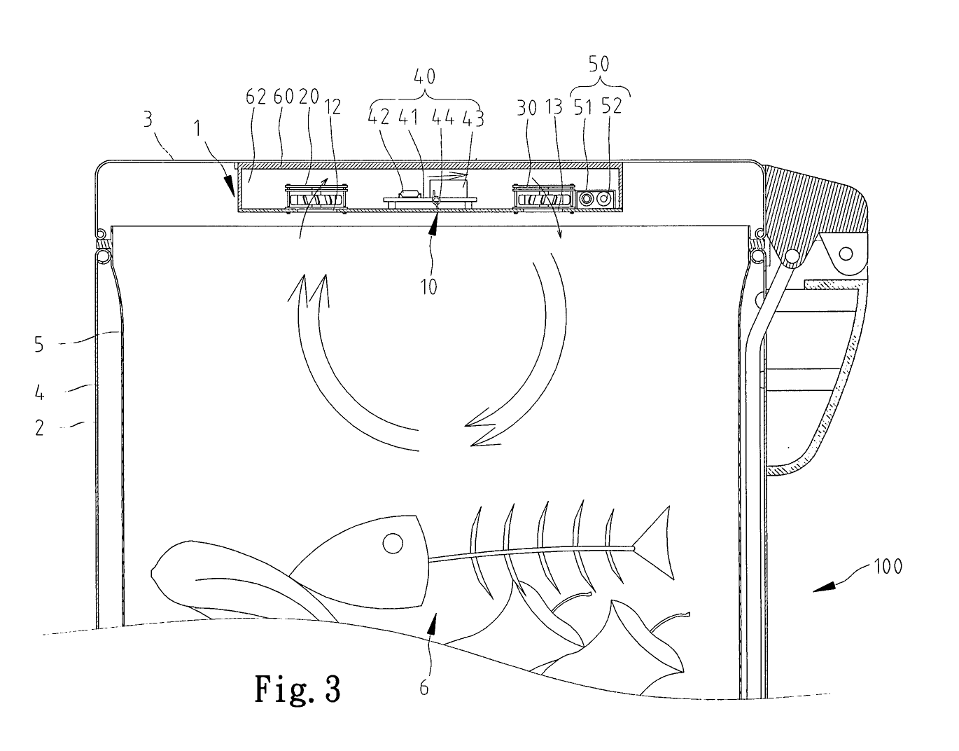

Referring to FIG. 3, kitchen waste is stored in the bin 2, and the bin 2 is closed by means of the cover 3. The ozone-based air cleaner 1 is activated. The first fan 20 draws air into the space defined by means of the board 10 and the shell 60 from the space defined in the bin 2 through the first passage 12. The second fan 30 draws air into the space defined in the bin 2 from the space defined by means of the board 10 and the shell 60 through the second passage 13. Driven by means of the first fan 20 and the second fan 30, an air current blows past the discharger 44 that generates ozone from the air by means of discharge. Air mixed with odor coming from the kitchen waste is cleaned by means of the resultant ozone. Therefore, when the cover 3 is lifted, no stinky smell bursts from the bin 2.

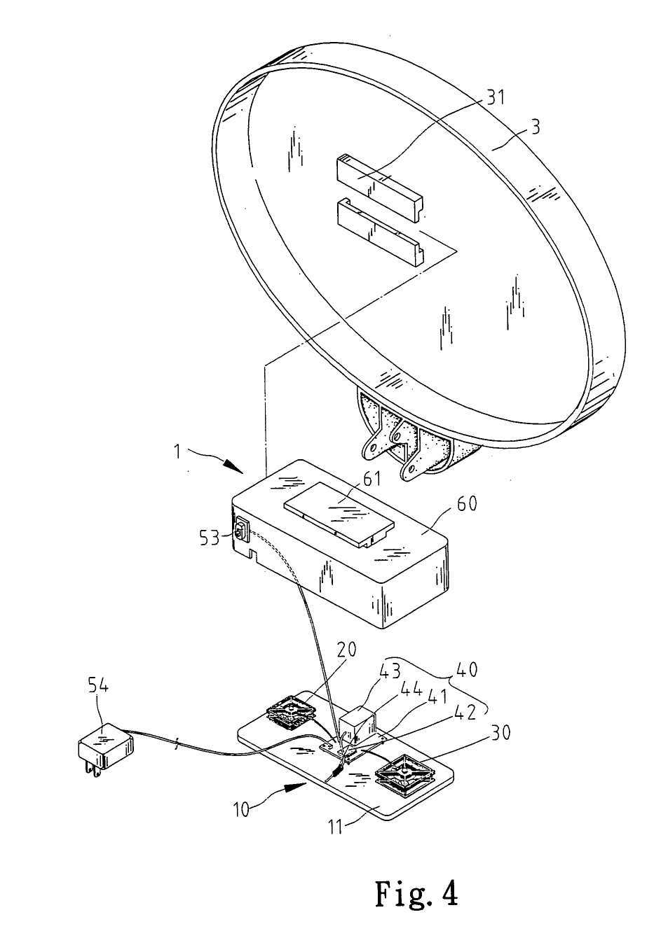

FIG. 4 shows a garbage storage apparatus according to a second embodiment of the present invention. The second embodiment differs from the first embodiment in two aspects. Firstly, a transformer 54 is used instead of the DC power supply 50. The transformer 54 is connected with the circuit board 41 through a wire (not numbered). Secondly, the switch 53 is connected with the circuit board 41 instead of the DC power supply 50. The switch 53 is connected with the circuit board 41 through a wire (not numbered).

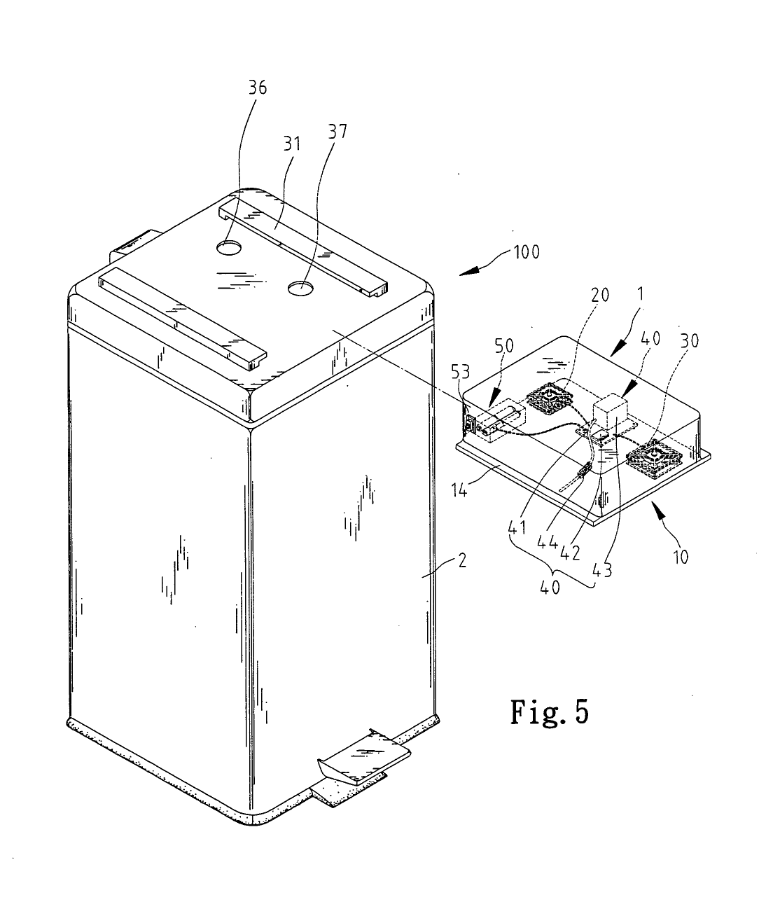

FIG. 5 shows a garbage storage apparatus according to a third embodiment of the present invention. The third embodiment differs from the third embodiment in four aspects. Firstly, the flanges 31 extend on an upper face instead of the lower face of the cover 3. Secondly, the cover 3 defines a first hole 36 and a second 37. The first hole 36 is communicated with the first passage 12, and the second hole 37 the second passage 13. Thirdly, the flanges 31 are engaged with two edges 14 of the board 10 instead of the rail 61. Fourthly, the rail 61 is saved.

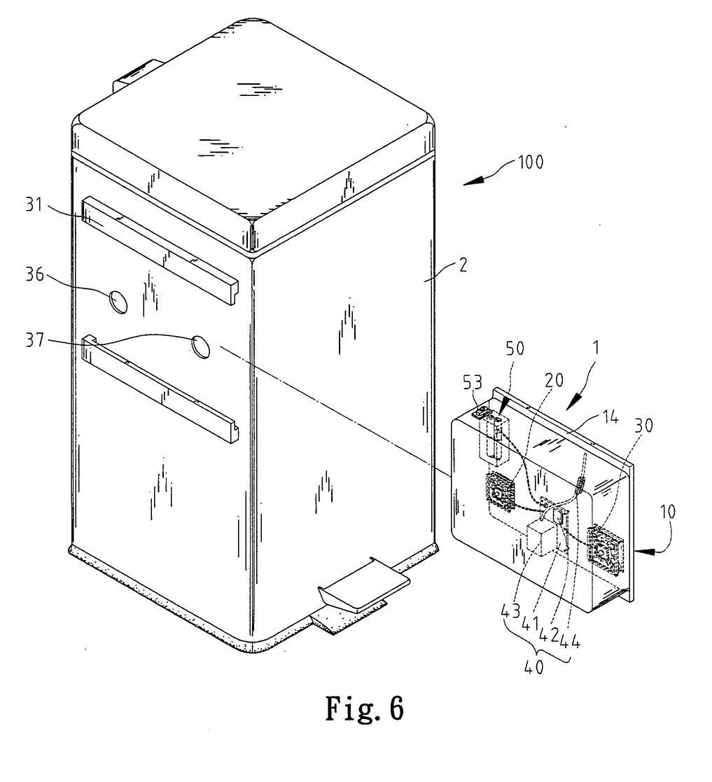

FIG. 6 shows a garbage storage apparatus according to a fourth embodiment of the present invention. The fourth embodiment differs from the third embodiment in two aspects. Firstly, the flanges 31 extend on an external face of the bin 2 instead of the upper face of the cover 3. Secondly, the first hole 36 and the second hole 37 are defined in the bin 2 instead of the cover 3.

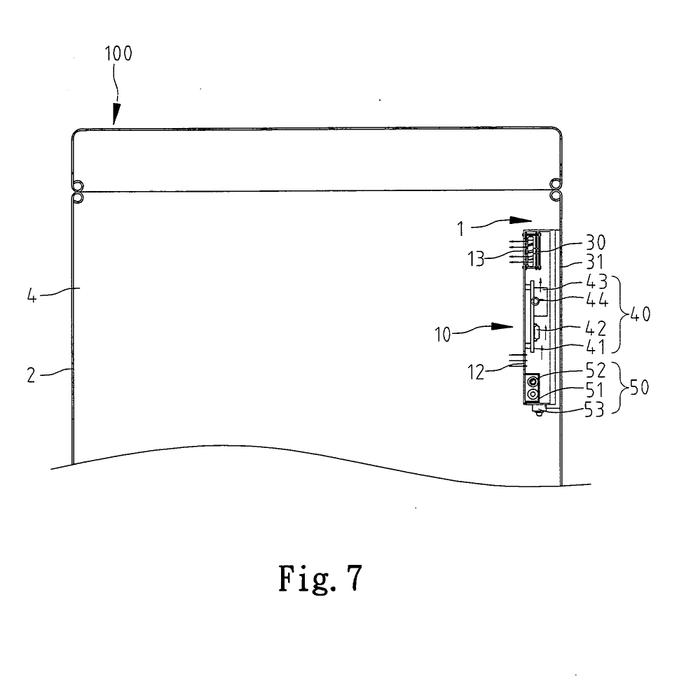

FIG. 7 shows a garbage storage apparatus according to a fifth embodiment of the present invention. The fifth embodiment differs from the fourth embodiment in two aspects. Firstly, the flanges 31 extend on an internal face instead of the external face of the bin 2. Secondly, the first hole 36 and the second hole 37 are saved.

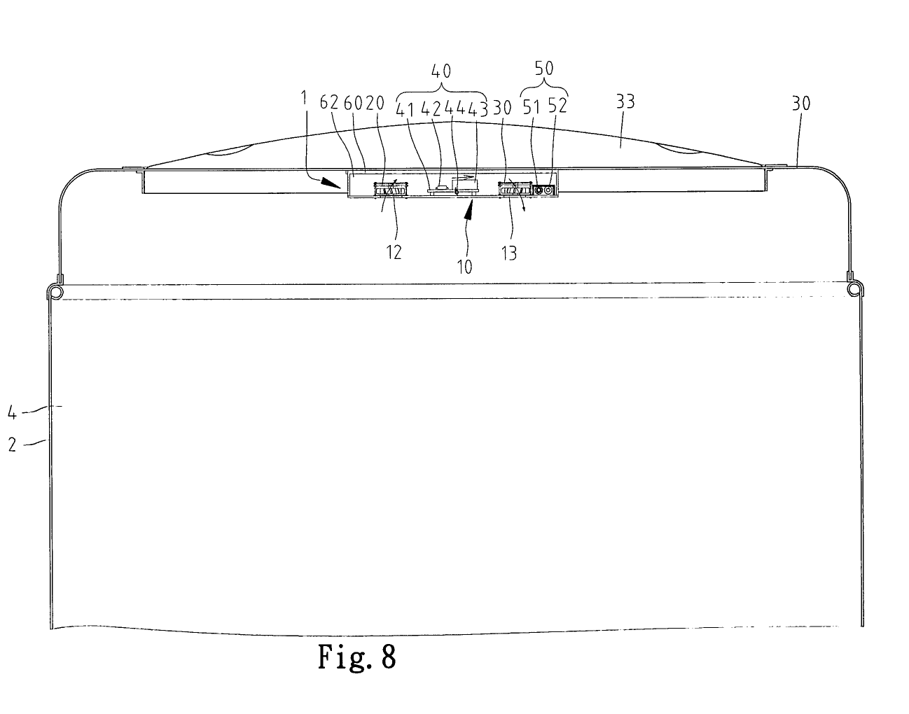

FIG. 8 shows a garbage storage apparatus according to a sixth embodiment of the present invention. The sixth embodiment differs from the first embodiment in two aspects. Firstly, a ring 30 and a cover 33 are used instead of the cover 3. The ring 30 is put on the bin 2. The cover 33 is rotationally mounted on the ring 30. Secondly, the ozone-based air cleaner 1 is secured to a lower face of the cover 30 instead of the lower face of the cover 3.

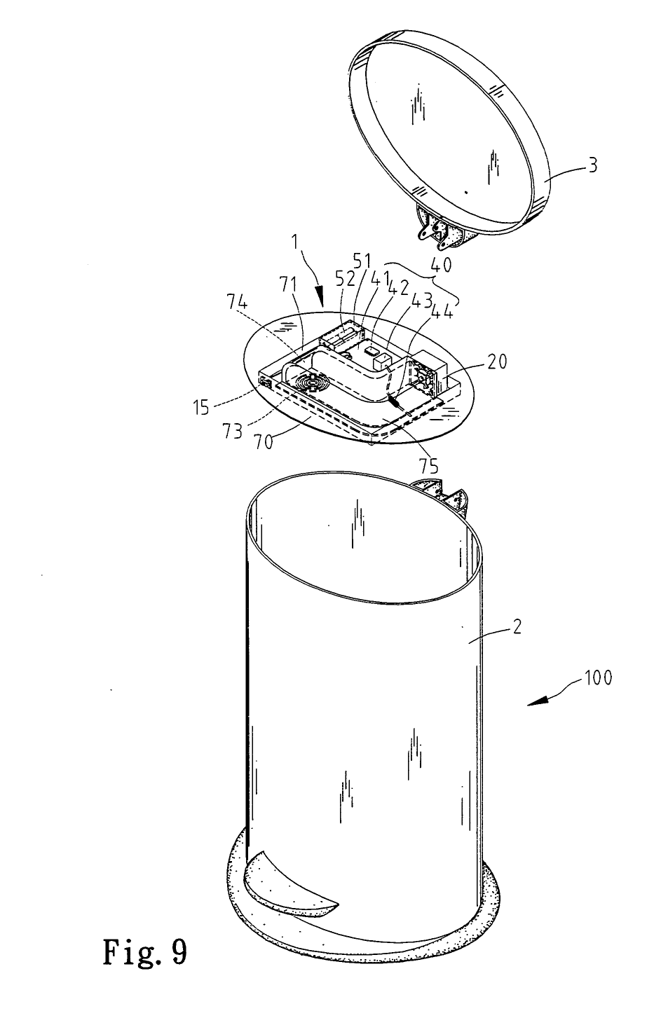

FIGS. 9˜11 show a garbage storage apparatus according to a seventh embodiment of the present invention. The seventh embodiment differs from the first embodiment in three aspects. Firstly, a tray 70 is used instead of the board 10. The tray 70 defines a cavity 71 in which the first fan 20, the second fan 30, the ozone generator 40 and the DC power supply 50 are put. The tray 70 defines a first passage 72 similar to the first passage 12 and a second passage 73 similar to the second passage 13. Secondly, a shell 74 is used instead of the shell 60. The shell 74 and the tray 70 define a channel between the first passage 72 and the second passage 73. Thirdly, the cover 3 is connected with the tray 70 instead of the shell 60.

The present invention has been described through detailed illustration of the preferred embodiment. Those skilled in the art can derive variations from the preferred embodiment without departing from the scope of the present invention. Therefore, the preferred embodiment shall not limit the scope of the present invention defined in the claims.

What is claimed is:

1. A garbage storage apparatus including:
a bin defining a space for storing garbage;
a cover for covering the bin; and
an ozone-based air cleaner communicated with the bin for cleaning air in the bin.

2. The garbage storage apparatus according to claim 1 wherein the ozone-based air cleaner includes an ozone generator for generating ozone from the air for cleaning the air.

3. The garbage storage apparatus according to claim 2 wherein the ozone generator includes a circuit board, an integrated circuit installed at the circuit board, a DC generator installed at the circuit board and a discharger connected with the DC generator for generating ozone by means of discharge.

4. The garbage storage apparatus according to claim 1 including a DC power supply connected with the ozone-based air cleaner.

5. The garbage storage apparatus according to claim 1 including a transformer connected with the ozone-based air cleaner.

6. The garbage storage apparatus according to claim 1 wherein the cover is pivotally mounted on the bin.

7. The garbage storage apparatus according to claim 1 including a ring arranged between the bin and the cover.

8. The garbage storage apparatus according to claim 1 wherein the ozone-based air cleaner is attached to the cover.

9. The garbage storage apparatus according to claim 8 wherein the ozone-based air cleaner is attached to the cover in a sliding manner.

10. The garbage storage apparatus according to claim 9 wherein the ozone-based air cleaner includes a rail formed thereon, and the cover includes two flanges formed thereon for engagement with the rail.

11. The garbage storage apparatus according to claim 9 wherein the ozone-based air cleaner includes a board, and the cover includes two flanges formed thereon for engagement with two edges of the board.

12. The garbage storage apparatus according to claim 8 wherein the ozone-based air cleaner is attached to an internal face of the cover.

13. The garbage storage apparatus according to claim 8 wherein the ozone-based air cleaner is attached to an external face of the cover.

14. The garbage storage apparatus according to claim 13 wherein the ozone-based air cleaner defines a first passage and a second passage, and the cover defines a first hole communicated with the first passage and a second hole communicated with the second passage.

15. The garbage storage apparatus according to claim 1 wherein the ozone-based air cleaner is attached to the bin.

16. The garbage storage apparatus according to claim 15 wherein the ozone-based air cleaner is attached to the bin in a sliding manner.

17. The garbage storage apparatus according to claim 16 wherein the ozone-based air cleaner includes a rail formed thereon, and the bin includes two flanges formed thereon for engagement with the rail.

18. The garbage storage apparatus according to claim 16 wherein the ozone-based air cleaner includes a board, and the bin includes two flanges formed thereon for engagement with two edges of the board.

19. The garbage storage apparatus according to claim 15 wherein the ozone-based air cleaner is attached to an internal face of the bin.

20. The garbage storage apparatus according to claim 15 wherein the ozone-based air cleaner is attached to an external face of the bin.

21. The garbage storage apparatus according to claim 20 wherein the ozone-based air cleaner defines a first passage and a second passage, and the bin defines a first hole communicated with the first passage and a second hole communicated with the second passage.
